(19) United States
(12) Patent Application Publication
(10) Pub. No.: US 2023/0260658 A1
(43) Pub. Date: Aug. 17, 2023

(54) POLYGENIC TRAIT PREDICTION USING LOCAL ANCESTRY

(71) Applicant: Myriad Genetics, Inc., Salt Lake City, UT (US)

(72) Inventors: Dmitry Pruss, Salt Lake City, UT (US); Alexander Gutin, Salt Lake City, UT (US); Elisha Hughes, Salt Lake City, UT (US); Jerry Lanchbury, Salt Lake City, UT (US)

(73) Assignee: Myriad Genetics, Inc., Salt Lake City, UT (US)

(21) Appl. No.: 17/920,013

(22) PCT Filed: Apr. 16, 2021
(86) PCT No.: PCT/US2021/027817

§ 371 Date: Oct. 19, 2022

Related U.S. Application Data
(60) Provisional application No. 63/012,699, filed on Apr. 20, 2020.

(51) Int. Cl. G16H 50/30; G16B 20/20; G16B 20/40; G16B 40/00

(52) U.S. Cl. CPC G16H 50/30; G16B 20/20; G16B 20/40; G16B 40/00

(57) ABSTRACT

Provided herein are methods for determining polygenic traits and risks for medical use, such as cancer traits and risks, as well as treating diseases for which risk is identified and/or assessed. Methods of this invention can provide a polygenic score which takes into account local ancestry through the ancestral origin of the alleles of risk loci. A polygenic score can provide surprisingly increased accuracy in determining polygenic traits and risks for ancestrally admixed populations.

### TECHNICAL FIELD

This invention relates to the fields of genetics and medicine. More particularly, this invention relates to methods for predicting polygenic traits and risks for medical use, as well as treating diseases for which risk can be assessed.

### BACKGROUND

It is desirable to use genomic measurements for determining the extent or manifestation of various biological traits. Recently, genotyping biological traits has involved predicting the risk of a clinical condition. Methods have involved genotyping polymorphic loci and determining a polygenic risk score to characterize the expectation of a clinical condition.

It is also desirable to use such polygenomic risk scores to assess the expectation of a clinical condition regardless of ancestry. However, risk scores from genomic data depend on identifying polymorphic loci to be used. Further, polygenic risk scores are specific to the particular population used for the measurements, and therefore are subject to the ancestries within the population.

A drawback of conventional methods for producing polygenic risk scores is that polymorphic loci identified in a particular population do not provide accurate polygenic risk scores in a different population.

For example, polymorphic loci identified in populations of European origin do not provide accurate polygenic risk scores in understudied and/or genetically diverse groups, including ancestrally admixed populations, such as those having both African and Eurasian ancestries. More particularly, polymorphic loci identified in populations of European origin do not provide accurate polygenic risk scores for US African-American and US Latin-American groups.

One way to utilize mixed-ancestry populations for identifying polymorphic loci has been to censor certain admixed subjects from the defining population. However, this creates drawbacks in diminishing the available information and accuracy of scores.

Another way is to adjust the eventual scores for the ancestral composition of the subjects of the population. Unfortunately, this creates drawbacks in relying on assumed genotype characteristics.

In general, conventional methods only take into account overall ancestral compositions of the study subjects, which does not provide accurate scores.

What is needed is an efficient and accurate method for determining polygenic risk scores with increased accuracy and reduced errors in predictive ability. An advantageous clinical risk algorithm can improve medical care and patient treatment.

There is an urgent need for methods to assess risk of a clinical condition such as cancer. There is a need for methods that can be efficiently brought to the point of medical care.

### BRIEF SUMMARY

This invention provides methods for determining polygenic traits and risks for medical use, as well as treating diseases for which risk is identified and/or assessed.

In some aspects, methods of this invention may provide superior prediction of clinical risk in patients harboring any diverse and/or mixed-origin ancestry. The methods of this invention can provide polygenic risk prediction which does not rely on self-reporting of ancestry by patients. Further, the methods of this invention can provide polygenic risk prediction which does not rely on so-called “genetic” ancestry composition.

In certain aspects, the clinical utility of this invention includes superior prediction of clinical risk in the patients harboring non-European ancestry, including African American patients, and patients of arbitrary mixed ancestry, and Latin American and European American ancestry with partial African genetic roots, in a manner not restricted by nor reliant on either self-reporting of the ancestry by the patients or the so-called genetic ancestry composition.

In some aspects, methods of this invention can provide a polygenic score which takes into account local ancestry. Local ancestry can be taken into account through the ancestral origin of the alleles of risk loci.

A polygenic score obtained by the methods of this invention can provide surprisingly increased accuracy in determining polygenic traits and risks for ancestrally admixed populations.

Examples of a polygenic trait include likelihood of cancer, such as breast cancer, and other diseases.

In further aspects, determining polygenic traits and risks can encompass identifying and utilizing genomic risk loci. Genomic risk loci may be associated with a trait, even though the genomic risk loci may be indirectly related to genomic effects and traits.

In some embodiments of this invention, an indirect-effect genomic risk loci may be associated with a trait only in a particular or local ancestral group.

Methods of this invention can provide surprisingly accurate determination of polygenic traits and risks by assessing and including contributions of local ancestral groups.

Embodiments of this invention contemplate determining the levels of polygenic traits and risks in the form of a score based on various genomic risk loci. The genomic risk loci can be discretely identified and defined, so that accurate determination can be done by genotyping subjects.

In certain aspects, the genomic risk loci can include genomic risk markers for a particular trait, which are combined with additional risk markers that can be ancestry-informative. The ancestry-informative markers may be adjacent and/or flanking certain genomic risk markers. The ancestry-informative markers can provide information on the contributions of local ancestral groups.

In certain embodiments, a score for a polygenic trait and/or risk can include determining the weights of additional ancestry-informative risk markers to be combined with genomic risk markers.

Embodiments of this invention include:

A method for assessing a biological trait in a subject, the method comprising:
measuring a genotype in a sample from the subject, the genotype having windows centered on trait risk markers for the trait, wherein the windows comprise additional ancestry-informative markers flanking the risk markers;
phasing the genotype to determine haplotypes in each window using reference populations having admixed ancestries;
calculating the odds of a local ancestral origin for each window; and
calculating a polygenic risk score for the biological trait in the subject using the trait risk markers and the odds of a local ancestral origin for each window, wherein the score is adjusted according to the local ancestral origins of the windows.

The number of trait risk markers can be from 1-10,000. Calculating the odds of a local ancestral origin may comprise dividing the phased genotypes within each window into consecutive non-overlapping tiles of up to about 300 additional ancestry-informative markers each, and calculating the odds of ancestral origin of each of the haplotypes in each tile using empirical frequencies of haplotypes in the reference population. Each tile may comprise 1-100 of the additional ancestry-informative markers. Each tile may comprise 5-20 of the additional ancestry-informative markers. The windows can be about 1 MB in width. The genotype can be determined by NGS. The genotype can be determined with a sequencing chip. The biological trait may be cancer likelihood. The genomic risk markers may be cancer markers. The additional ancestry-informative markers can be SNP markers or indel markers. The genomic risk markers may be breast cancer SNP markers. Calculating a polygenic risk score may comprise calculating incremental contributions of each allele to the polygenic risk score as a local ancestry-specific risk effect beta multiplied by a number of risk alleles genotyped, which is zero or 1, less a population-specific risk allele frequency.

This invention further includes methods for recommending therapy for a subject having a disease, the method comprising:
measuring a genotype in a sample from the subject, the genotype comprising risk markers associated with the trait, and further comprising additional ancestry-informative markers flanking the risk markers;
phasing the genotype to determine haplotypes in each window with reference populations having admixed ancestries;
calculating the odds of a local ancestral origin for each window; and
calculating a polygenic risk score for the biological trait in the subject using the trait risk markers and the odds of a local ancestral origin for each window, wherein the score is adjusted according to the local ancestral origins of the windows; and
recommending a therapy for the disease based on the risk score exceeding a threshold level. The disease can be cancer, or breast cancer. The therapy may be one of a therapy for the disease; a monitoring period followed by a therapy for the disease; or a tapering of a therapy for the disease. The therapy can be one or more of surgery, cryoablation, radiation therapy, bone marrow transplant, chemotherapy, immunotherapy, hormone therapy, stem cell therapy, drug therapy, biological therapy, and administration of a pharmaceutical, prophylactic or therapeutic compound.

This invention further includes methods for identifying a subject having a disease who benefits from a treatment, the method comprising:
measuring a genotype in a sample from the subject, the genotype comprising risk markers associated with the trait, and further comprising additional ancestry-informative markers flanking the risk markers;
phasing the genotype to determine haplotypes in each window with reference populations having admixed ancestries;
calculating the odds of a local ancestral origin for each window;
calculating a polygenic risk score for the biological trait in the subject using the trait risk markers and the odds of a local ancestral origin for each window, wherein the score is adjusted according to the local ancestral origins of the windows; and
identifying the subject having the disease who benefits from a treatment for the disease based on the risk score indicating a need for a treatment, or exceeding a threshold level. The disease may be cancer, or breast cancer.

Embodiments of this invention further contemplate methods for treating a disease in a subject in need thereof, the method comprising:
measuring a genotype in a sample from the subject, the genotype comprising risk markers associated with the trait, and further comprising additional ancestry-informative markers flanking the risk markers;
phasing the genotype to determine haplotypes in each window with reference populations having admixed ancestries;
calculating the odds of a local ancestral origin for each window;
calculating a polygenic risk score for the biological trait in the subject using the trait risk markers and the odds of a local ancestral origin for each window, wherein the score is adjusted according to the local ancestral origins of the windows; and
administering to the subject one of: a therapy for the disease; a monitoring period followed by a therapy for the disease; or a tapering of a therapy for the disease.

Additional embodiments include methods for monitoring a response of a subject having a disease, the method comprising:
measuring a genotype in a sample from the subject, the genotype comprising risk markers associated with the trait, and further comprising additional ancestry-informative markers flanking the risk markers;
phasing the genotype to determine haplotypes in each window with reference populations having admixed ancestries;
calculating the odds of a local ancestral origin for each window;
calculating a polygenic risk score for the biological trait in the subject using the trait risk markers and the odds of a local ancestral origin for each window, wherein the score is adjusted according to the local ancestral origins of the windows.

This invention includes methods for prognosing a subject having a disease, the method comprising:
measuring a genotype in a sample from the subject, the genotype comprising risk markers associated with the trait, and further comprising additional ancestry-informative markers flanking the risk markers;
phasing the genotype to determine haplotypes in each window with reference populations having admixed ancestries;
calculating the odds of a local ancestral origin for each window;
calculating a polygenic risk score for the biological trait in the subject using the trait risk markers and the odds of a local ancestral origin for each window, wherein the score is adjusted according to the local ancestral origins of the windows; and
prognosing the subject as having a poor prognosis for the disease based on the risk score, the risk score indicating a need for a therapy, or exceeding a threshold level.

Further embodiments include a system for assessing risk of a disease in a subject, the system comprising:
a processor for receiving genomic data from a sample of the subject;
one or more processors for carrying out the steps:
   measuring a genotype in a sample from the subject, the genotype having windows centered on trait risk markers for the trait, wherein the windows comprise additional ancestry-informative markers flanking the risk markers;
   phasing the genotype to determine haplotypes in each window using reference populations having admixed ancestries;
   calculating the odds of a local ancestral origin for each window; and
   calculating a polygenic risk score for the biological trait in the subject using the trait risk markers and the odds of a local ancestral origin for each window; and
a display for displaying and/or reporting the risk score.

Also included is a non-transitory machine-readable storage medium having stored therein instructions for execution by a processor which cause the processor to perform the steps of a method for assessing risk of a disease in a subject, the method comprising:
receiving genomic data from a sample from the subject;
measuring a genotype in the sample, the genotype having windows centered on trait risk markers for the trait, wherein the windows comprise additional ancestry-informative markers flanking the risk markers;
phasing the genotype to determine haplotypes in each window using reference populations having admixed ancestries;
calculating the odds of a local ancestral origin for each window;
calculating a polygenic risk score for the biological trait in the subject using the trait risk markers and the odds of a local ancestral origin for each window; and
sending to a processor output for displaying and/or reporting the risk score.

### BRIEF DESCRIPTION OF THE DRAWINGS

FIG. 1 shows tiling a sub-centiMorgan area into short local haplotypes. Haplotype frequencies can be used from a public dataset to accurately separate local African vs. European ancestry. 9 windows of 12 markers each are shown. The likelihood of African local ancestry in African-American (AfrAm).

FIG. 2 shows that the IBS (Iberian / Spain) dataset, part of the 1000 Genomes (G1K) Europeans, exhibits up to 2-3 African haplotypes per locus in local ancestry mapping. Some European minor allele carriers in G1K seem to have very African-like linkage disequilibrium (LD) patterns. Eur (dotted line), Afr (solid line).

FIG. 3 shows local/global ancestry distributions and AFs are as expected. Distribution of predicted ethnic origin for rs132390. Empirical AfrAm (dotted line), G1K Afr (solid line), G1K Eur (dashed line).

FIG. 4 shows European global ancestry averages of 17% with a wide distribution, as expected.

FIG. 5 shows empirical MAFs from European local ancestry segments (y axis) vs. G1K MAF values (x axis).

FIG. 6 shows empirical MAFs from African local ancestry segments (y axis) vs. G1K MAF values (x axis).

FIG. 7 shows a number of markers may be needed for Eur/Afr local ancestry assignment. A relatively small number of flanking markers (x-axis) can be used to achieve surprisingly high 90% to 95% accuracy of local Afr/Eur ancestry identification. 95% accuracy (dashed line), 90% accuracy (solid line).

FIG. 8 shows a number of markers may be needed for Eur/EAS local ancestry assignment. A relatively small number of flanking markers (x-axis) can be used to achieve surprisingly high 90% to 95% accuracy of separating European from East Asian ancestry identification. 95% accuracy (dashed line), 90% accuracy (solid line).

FIG. 9 shows an error rate of local ancestry assignment with unphased genotypes. FIG. 9 shows results with 3 SNP loci, rs258809, rs10759243, and rs1550623, and the y-axis displays the error rates of local ancestry deconvolution depending on the number of flanking loci.

### DETAILED DESCRIPTION OF THE DISCLOSURE

This invention includes methods for using local ancestry in polygenic risk prediction to provide accurate risk assessment for all subjects, regardless of ancestry.

Embodiments of this invention further provide reliable trait associations in ancestrally diverse and genetically admixed populations.

This disclosure provides various methods for association studies for multiple candidate loci, which may be characterized by a broader linkage disequilibrium (LD) pattern. Most, or all, do not exert direct effects on the trait.

Embodiments of this invention can provide clinical risk management, risk magnitude assessment, as well as polygenic risk scores, and non-clinical trait prediction. Methods of this invention can provide predictive ability that is accurate for all subjects, even admixed genotypes.

Aspects of this disclosure include genotyping polymorphic loci and combining the genotypes in the form of a polygenic score to predict risk of a clinical condition or an extent of manifestation of a biological trait.

In some embodiments, a polygenic score prediction may be specific to the populations and ancestries within which they were found.

In further embodiments, a plurality of trait risk markers can be used along with additional ancestry-informative markers to provide a polygenic risk prediction for the trait.

In certain embodiments, the plurality of trait risk markers may be from 1-10,000 markers, or from 1-1000 markers, or from 1-100 markers.

Some risk markers can appear in a window of the genotype. The window can be about ± 0.5 MB, which is a 1 MB window, or ± 1 MB, or ± 2 MB. In certain embodiments, the window can be any size useful for phasing the genotype.

This invention can provide risk prediction portability for understudied and genetically diverse groups. In certain embodiments, this invention can provide risk prediction portability for ancestrally admixed populations such as those harboring both African and Eurasian ancestries, including African Americans and Latin Americans in the US.

In further aspects, provided herein are methods for portable prediction of trait associations between divergent populations. Embodiments herein can provide improved polygenic risk prediction even in the presence of the indirect nature of the allele effects on the trait. In certain embodiments, various direct-effect loci may remain undiscovered, but can be bound to the discovered loci by linkage disequilibrium. Various direct-effect loci may be separated by genetic distances on the order of a centimorgan.

Aspects of this invention can provide unique methods for identifying each allele of a genotype with its ancestry.

Further aspects of this invention can provide unique methods for determining a polygenic risk score for predicting a biological trait.

In some aspects, large size association studies can be used with greater power to break up linkage disequilibrium (LD) patterns, and to focus on fewer loci. In some embodiments, a single best candidate SNP may not be a true direct-effect allele.

In further aspects, an LD pattern between a candidate SNP and a true direct-effect allele may be retrieved in populations of different ancestry.

In certain aspects, one or more risk SNPs can be discovered in European populations. The risk SNPs may retain significance in East Asian populations, and may have reduced odds ratio. Some European SNPs can be used for African populations.

In some embodiments, the genetic-distance resolution of GWAS may be in the 0.1 cM range, which means that the LD patterns between candidate SNPs and true risk loci are expected to fade out a few 1000′s generations ( 20,000 to 60,000 years). For example, Eurasian populations may share substantial common ancestry in this time frame with one another, but not with the Africans.

In further embodiments, genetic ancestry, both local and global, can be used with similar alleles, for different, indirect, effects. The alleles identified by GWAS studies are rarely unique to one ancestral population. However, both odds ratios and average score values may vary widely from ancestry to ancestry. The gaps between cultural or self-reported and genetic ancestry can be substantial. Genetic ancestry may accurately inform the SNP-trait effect size.

In certain embodiments, when a population is historically recently admixed, with the characteristic ancestral chromosomal blocks >> 0.1 cM, then only local ancestry of the DNA segments can inform the effect calculation.

For African American local ancestry, the predominant source populations may be West African, over 80% on average, with wide variation, and European, under 20%. The average European segment size may be near 30 cM, with many shorter segments, which a low marker density study might mix.

This invention contemplates calculating local ancestry in phased genomes.

In some embodiments, local haplotypes can be ancestry-informative.

In further aspects, this invention provides tiling of a sub-centiMorgan area into short local haplotypes. In certain aspects, a haplotype frequency can be used from a reference dataset to accurately separate local African vs. European ancestry.

Embodiments of this invention can make millennia-old admixtures come into light. Ancestral reference populations may have themselves experienced limited admixing in the historical, ≥ 0.1 cM, timeframe. Low-level African admixture in the Iberian peninsula may be observed, and can be timed to Roman, and, partly, Kalifate times by ancient DNA studies. An IBS (Iberian / Spain) reference dataset may be a part of the 1000 Genomes Europeans, and may provide up to 2-3 African haplotypes per locus in local ancestry mapping.

In some embodiments, a high-density gene array can be used. The array may include from 2 x 10⁵ to 5 x 10⁶ SNPs.

In certain embodiments, a high-density AXIOM array can be used.

In further embodiments, breast cancer risk markers can be used along with additional flanking SNPs in windows. The windows can be about ± 0.5 MB, which is a 1 MB window.

Some examples of breast cancer risk markers are given in: Prediction of breast cancer risk based on profiling with common genetic variants, Mavaddat et al., J Natl Cancer Inst., 2015, April 8, Vol. 107(5).

Some examples of breast cancer risk markers are given in Characterizing Genetic Susceptibility to Breast Cancer in Women of African Ancestry, Feng et al., Cancer Epidemiol Biomarkers Prev., 2017, July, Vol. 26(7), pp. 1016-1026.

Some examples of breast cancer risk markers are given in Early Diagnosis of Breast Cancer, Wang et al., Sensors (Basel), 2017, July, Vol. 17(7), p. 1572.

In certain aspects, genotypes can be phased with Beagle 5.1, using African and European reference datasets of the 1000 Genomes project. The phased genotypes may be divided into non-overlapping tiles of about 15 markers each. The odds of African vs. European origin can be calculated for each tile.

In further aspects, a global ancestry-corrected polygenic risk score may be surprisingly more accurate than an average-ancestry corrected polygenic risk score.

In some aspects, a local ancestry-corrected polygenic risk score can be surprisingly more accurate than a global ancestry-corrected polygenic risk score.

In further aspects, an association between the polygenic risk scores and breast cancer may be evaluated by logistic regression. The logistic regression may be adjusted for age and family history, among other variables. A logistic regression may include parameters or data for age and family history, among other variables.

In additional aspects, flanking markers can be added one by one from the phased 1000 Genomes genotypes, for maximizing local ancestry separation at each iteration.

In certain aspects, an advantageously small number of flanking markers can be used to achieve 90 to 95% accuracy of local African / European ancestry identification.

In further embodiments, the difficult task of separating European from East Asian ancestry may require less than about 20 flanking additional SNP markers.

### Polygenic Risk Scores

Aspects of this invention contemplate methods for calculating polygenic scores which take into account the ancestral origin of the alleles of the risk loci, namely local ancestry.

Methods of this invention can use any number of trait risk markers. Methods for determining a local ancestry-corrected polygenic risk score of this invention may use any 3 or more markers, or any 5 or more markers, or any 10 or more markers of a set of markers shown in Table 2.

Aspects of this invention contemplate methods for calculating polygenic scores with adjustment for the ancestral origin of the alleles of the risk loci, namely local ancestry.

In some embodiments, local ancestry adjustment may dramatically improve polygenic score portability in predicting traits such as breast cancer in ancestrally admixed populations.

In further embodiments, indirect-effect risk loci can be associated with a trait only in one of the ancestral groups. In other embodiments, a risk allele may have direct effects which can be similar in size across the spectrum of local ancestries.

Embodiments of this invention provide methods for accurately gauging the local ancestry-dependent contributions of the alleles to the risk prediction. The methods of this invention can provide risk prediction with surprisingly increased accuracy over conventional adjustments based on overall ancestral compositions of the study subjects.

Methods of this disclosure can include steps for identifying ancestral origins of all of the clinically or trait-relevant genotypes.

In certain embodiments, genotypes of numerous additional ancestry-informative loci which are adjacent to the score-able loci can be used.

In further embodiments, specific weights can be applied to score-able loci depending on their ancestral origin, namely local ancestry.

Steps of a method of this invention may include genotyping anonymized African American subjects. Genotyping can be done by any method, including NDS, a custom chip, or a combination of NGS and a chip.

In some embodiments, a subset of known breast cancer risk markers can be used along with additional markers in a window centered on each known risk marker. The subset may have higher allelic frequencies in the public African and European controls, as well as lower linkage disequilibrium among the additional markers.

Examples of additional markers include SNPs, and indels.

In certain embodiments, the genotypes can be haplotype phased. Genotype phasing can be done by known methods.

Examples of methods for haplotype estimation include hidden Markov model (HMM), PHASE, Gibbs sampling, fastPHASE, BEAGLE, haplotype cluster modelling, IMPUTE2, MaCH, SHAPEIT1, HAPI-UR, and SHAPEIT2.

In additional embodiments, genotype phasing may be done with mismatches, based on a similarity factor.

In further steps, methods for haplotype estimation may use African and European reference datasets.

Examples of reference data sets include the 1000 Genomes project. Examples of reference data sets include public and/or private collections of more than 10⁵ genomes.

In additional embodiments, a phased genotype window may be divided into consecutive non-overlapping tiles. Each tile can include a plurality of markers. In certain embodiments, a tile may contain up to about 300 markers, or from 1-100 markers, or from 2-50 markers, or from 5-40 markers, or from 5-20 markers.

Additional steps of a method of this invention may include calculating the odds of African vs. European origin of each of the haplotypes in each tile. In certain steps, calculating the odds of African vs. European origin of each of the haplotypes may be done using empirical frequencies of haplotypes in a reference sets. For a haplotype that is absent in a reference sets, a frequency of 0.5 observations per set may be used. A haplotype that is absent in all reference sets can be considered to be equally likely to be African or European.

In some embodiments, the odds of a locus being African vs. European in ancestral origin can be found according to Equation I.

$${p}_{A \left|E\right)} = {\prod }_{i = 1}^{K} \left(\frac{N \left({h}_{i} , A\right) ∗ N \left(E\right)}{N \left(A\right) ∗ N \left({h}_{i} , E\right)}\right) \qquad \text{Equation I.}$$

where K is the number of haplotype tiles, hᵢ is the haplotype at the tile i, N(hᵢ,A) and N(hᵢ,E) are the observed counts of the in the African (A) and European (E) reference sets, and N(E)/N(A) are the total numbers of haplotypes in the respective reference sets.

In further embodiments, the overall odds of African vs. European ancestral origin of the entire segment containing each of the alleles of a risk SNPs can be calculated as the product of odds of all tile haplotypes contained within the segment. Fractional probabilities of each allele’s being “ancestrally European” vs. “ancestrally African” can be assigned, being the fractional local ancestry, according to Equation II.

$$L A = 1 / \left({p}_{A \left|E\right)} + 1\right) \qquad \text{Equation II.}$$

In additional embodiments, the overall fraction of European ancestry of each study subject, namely the global ancestry GA, can be calculated as the average of fractional European local ancestry for all loci, according to Equation III.

$$G A = \frac{\left({\sum }_{l o c i} L A\right)}{{N}_{l o c i}} \qquad \text{Equation III.}$$

Further steps of a method of this invention may include calculating incremental contributions of each allele to the polygenic risk score as a local ancestry-specific risk effect, a beta, multiplied by a number of risk alleles genotyped, where a GT_{SNP} can be zero or 1, less population-specific risk allele frequency.

In some embodiments, a local ancestry ancestry-specific beta for European local ancestry may be known. For risk markers which failed to show significant and consistent effects in the African studies, the African ancestry-specific betas may be assumed to be equal to zero. A local ancestry ancestry-specific beta for African local ancestry may also be known, or can be assumed to be zero. In certain embodiments, for risk markers with marginally significant effects in African population study, the African ancestry-specific betas may be assumed to be scaled down betas from the European study.

In some aspects, a comparative conventional polygenic risk score (cPRS) may be calculated on the assumption that the respective betas of the ancestral groups β_{A,SNP} and β_{E,SNP} can be interpolated using weights equal to respective global ancestry averages, GAA for the European ancestry and (1-GAA) for the African ancestry. The conventional score may be centered on the admixed population-specific minor allele frequency MAF_{AA,SNP}, according to Equations IV and V.

$$\begin{matrix}c P R S = {\sum }_{S N P a l l e l e s} \left(\left(1 - G A A\right) ∗ {β}_{A , S N P} + G A A ∗ {β}_{E , S N P}\right) ∗ \\ \left(G {T}_{S N P} - M A {F}_{A A , S N P}\right)\end{matrix} \qquad \text{Equation IV}$$

where

$$G A A = \frac{\left({\sum }_{s a m p l e s} G A\right)}{{N}_{s a m p l e s}} \qquad \text{Equation V.}$$

In certain aspects, a comparative or conventional method can involve global ancestry adjustment, which may interpolate the ancestral betas using the actual global ancestry of each subject, according to Equation VI.

$$\begin{matrix}g l o b a l P R S = {\sum }_{S N P a l l e l e s} \left(\left(1 - G A\right) ∗ {β}_{A , S N P} + G A ∗ {β}_{E , S N P}\right) ∗ \\ \left(G {T}_{S N P} - M A {F}_{A A , S N P}\right)\end{matrix} \qquad \text{Equation VI.}$$

In further aspects, embodiments of this invention can provide methods for local ancestry adjustment, which may interpolate the ancestral betas using the fractional likelihood, LA, of each specific allele being European, and centering both ancestral components of the score on respective ancestral minor allele frequencies MAF_{A,SNP} and MAF_{E,SNP}, according to Equation VII.

$$\begin{matrix}l o c a l P R S = \\ {\sum }_{S N P a l l e l e s} \left(\left(1 - L A\right) ∗ {β}_{A , S N P} ∗ \left(G {T}_{S N P} - M A {F}_{A , S N P}\right) +\right) \\ \left(L A ∗ {β}_{E , S N P} ∗ \left(G {T}_{S N P} - M A {F}_{E , S N P}\right)\right)\end{matrix} \qquad \text{Equation VII.}$$

In certain aspects, an average African American population-specific beta may be calculated by interpolation between European and African local ancestry-specific betas using average percentages of European vs. African ancestries. The global ancestry-corrected betas can be calculated by interpolation between European and African local ancestry-specific betas using the actual percentage of European vs. African ancestry in each specific subject.

Methods of this invention can use any number of trait risk markers. Methods for determining a local ancestry-corrected polygenic risk score of this invention may use any 3 or more markers, or any 5 or more markers, or any 10 or more markers of a set of markers shown in Table 2.

### Cancer Scores and Treatment

Cancer therapy can include surgery, cryoablation, radiation therapy, bone marrow transplant, chemotherapy, immunotherapy, hormone therapy, stem cell therapy, drug therapy, biological therapy, and administration of a pharmaceutical, prophylactic or therapeutic compound including, for example, a biologic or exogenous active agent.

Examples of treatments include bariatric surgical intervention, physical therapy, diet, and diet supplementation.

Examples of a cancer biological therapy include adoptive cell transfer, angiogenesis inhibitors, bacillus Calmette-Guerin therapy, biochemotherapy, cancer vaccines, chimeric antigen receptor (CAR) T-cell therapy, cytokine therapy, gene therapy, immune checkpoint modulators, immunoconjugates, monoclonal antibodies, oncolytic virus therapy, and targeted drug therapy.

Examples of a cancer surgery include lumpectomy, partial mastectomy, total mastectomy, simple mastectomy, modified radical mastectomy, radical mastectomy, and Halsted radical mastectomy.

Examples of a cancer drug include drugs approved to prevent breast cancer including Evista (Raloxifene Hydrochloride), Raloxifene Hydrochloride, and Tamoxifen Citrate.

Examples of a cancer drug include drugs approved to treat breast cancer including, Abemaciclib, Abraxane (Paclitaxel Albumin-stabilized Nanoparticle Formulation), Ado-Trastuzumab Emtansine, Afinitor (Everolimus), Afinitor Disperz (Everolimus), Alpelisib, Anastrozole, Aredia (Pamidronate Disodium), Arimidex (Anastrozole), Aromasin (Exemestane), Atezolizumab, Capecitabine, Cyclophosphamide, Docetaxel, Doxorubicin Hydrochloride, Ellence (Epirubicin Hydrochloride), Enhertu (Fam-Trastuzumab Deruxtecan-nxki), Epirubicin Hydrochloride, Eribulin Mesylate, Everolimus, Exemestane, 5-FU (Fluorouracil Injection), Fam-Trastuzumab Deruxtecan-nxki, Fareston (Toremifene), Faslodex (Fulvestrant), Femara (Letrozole), Fluorouracil Injection, Fulvestrant, Gemcitabine Hydrochloride, Gemzar (Gemcitabine Hydrochloride), Goserelin Acetate, Halaven (Eribulin Mesylate), Herceptin Hylecta (Trastuzumab and Hyaluronidase-oysk), Herceptin (Trastuzumab), Ibrance (Palbociclib), Ixabepilone, Ixempra (Ixabepilone), Kadcyla (Ado-Trastuzumab Emtansine), Kisqali (Ribociclib), Lapatinib Ditosylate, Letrozole, Lynparza (Olaparib), Megestrol Acetate, Methotrexate, Neratinib Maleate, Nerlynx (Neratinib Maleate), Olaparib, Paclitaxel, Paclitaxel Albumin-stabilized Nanoparticle Formulation, Palbociclib, Pamidronate Disodium, Perjeta (Pertuzumab), Pertuzumab, Piqray (Alpclisib), Ribociclib, Talazoparib Tosylate, Talzenna (Talazoparib Tosylate), Tamoxifen Citrate, Taxotere (Docetaxel), Tecentriq (Atezolizumab), Thiotepa, Toremifene, Trastuzumab, Trastuzumab and Hyaluronidase-oysk, Trexall (Methotrexate), Tykerb (Lapatinib Ditosylate), Verzenio (Abemaciclib), Vinblastine Sulfate, Xeloda (Capecitabine), and Zoladex (Goserelin Acetate).

As used herein, the term “disease” includes any disorder, condition, sickness, ailment that manifests in, for example, a disordered or incorrectly functioning organ, part, structure, or system of the body.

As used herein, the term “sample” includes any biological sample that is isolated from a subject. A sample can include, without limitation, a single cell or multiple cells, fragments of cells, an aliquot of body fluid, whole blood, platelets, serum, plasma, red blood cells, white blood cells or leucocytes, endothelial cells, tissue biopsies, synovial fluid, lymphatic fluid, ascites fluid, and interstitial or extracellular fluid. The term “sample” also encompasses the fluid in spaces between cells, including synovial fluid, gingival crevicular fluid, bone marrow, cerebrospinal fluid (CSF), saliva, mucous, sputum, semen, sweat, urine, or any other bodily fluids. A blood sample can include whole blood or any fraction thereof, including blood cells, red blood cells, white blood cells or leucocytes, platelets, serum and plasma.

As used herein, the term “subject” includes humans, as well as mammals.

In some embodiments, this invention can provide methods for recommending therapeutic regimens, including withdrawal from therapeutic regiments.

In further embodiments, an odds ratio can provide a clinician with a prognostic picture of a subject’s biological state. Such embodiments may provide subject-specific prognostic information, which can be informative for a therapy decision, and may also facilitate monitoring therapy response. Such embodiments may result in a surprisingly improved treatment, such as better control of a disease, or an increase in the proportion of subjects achieving amelioration of symptoms.

As used herein, the terms “biologic,” “biotherapy,” and/or “biopharmaceutical” can include pharmaceutical therapy products manufactured or extracted from a biological substance. A biologic can include vaccines, blood or blood components, allergenics, somatic cells, gene therapies, tissues, recombinant proteins, and living cells; and can be composed of sugars, proteins, nucleic acids, living cells or tissues, or combinations thereof.

As used herein, the terms “therapeutic regimen,” “therapy” and/or “treatment” can include any clinical management of a subject, as well as interventions, whether biological, chemical, physical, or a combination thereof, intended to sustain, ameliorate, improve, or otherwise alter the condition of a subject.

As used herein, the term “administering” can include the placement of a composition into a subject by a method or route that results in at least partial localization of the composition at a desired site such that a desired effect is produced. Routes of administration include both local and systemic administration. Generally, local administration results in more of the composition being delivered to a specific location as compared to the entire body of the subject, whereas, systemic administration results in delivery to essentially the entire body of the subject. “Administering” also includes performing physical actions on a subject’s body, including physical therapy, as well as chiropractic treatment, massage and acupuncture.

### Devices and Systems

As used herein, the term machine-readable storage medium can comprise, for example, a data storage material that is encoded with machine-readable data or data arrays. The data and machine-readable storage medium may be capable of being used for a variety of purposes, when using a machine programmed with instructions for using said data. Such purposes include storing, accessing and manipulating information relating to the risk of a subject or population over time, or risk in response to treatment, or for drug discovery for inflammatory disease. Data comprising genomic measurements can be implemented in computer programs that are executing on programmable computers, which may comprise a processor, a data storage system, one or more input devices, one or more output devices. Program code can be applied to the input data to perform the functions described herein, and to generate output information. Output information can then be applied to one or more output devices. A computer can be, for example, a personal computer, a microcomputer, or a workstation.

As used herein, the term computer program can be instruction code implemented in a high-level procedural or object-oriented programming language, to communicate with a computer system. The program may be implemented in machine or assembly language. The programming language can also be a compiled or interpreted language. Each computer program can be stored on storage media or a device such as ROM, or magnetic diskette, and can be readable by a programmable computer for configuring and operating the computer when the storage media or device is read by the computer to perform the described procedures. A health-related or genomic data management system can be considered to be implemented as a computer-readable storage medium, configured with a computer program, where the storage medium causes a computer to operate in a specific manner to perform various functions.

### Conclusion

All publications, patents and literature specifically mentioned herein are hereby incorporated by reference in their entirety for all purposes.

Words specifically defined herein have the meaning provided in the context of the present disclosure as a whole, and as are typically understood by those skilled in the art. As used herein, the singular forms “a,” “an,” and “the” include the plural.

While the present disclosure is described in conjunction with various embodiments, it is not intended that the present disclosure be limited to such embodiments. On the contrary, the present disclosure encompasses various alternatives, modifications, and equivalents, as will be appreciated by those of skill in the art.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. In addition, the materials, methods, and examples herein are illustrative only and not intended to be limiting.

Although the foregoing disclosure has been described in some detail by way of illustration and examples for purposes of clarity of understanding, it will be understood by persons of skill in the art that various changes and modifications may be practiced within the scope of the invention and the appended claims.

### EXAMPLES

### Example 1: Genotype Windows for Markers

Polygenic risk scores were calculated with adjustment for the local ancestral origin of the alleles of the risk loci. To identify ancestral origins of all the clinically or trait-relevant genotypes, genotypes of a plurality of additional ancestry-informative loci adjacent to the score-able loci were used.

The genotypes were phased with Beagle 5.1 using African and European reference datasets of the 1000 Genomes project as a reference. Each 1 MB window of the phased genotypes was divided into consecutive non-overlapping tiles of 15 SNP markers each.

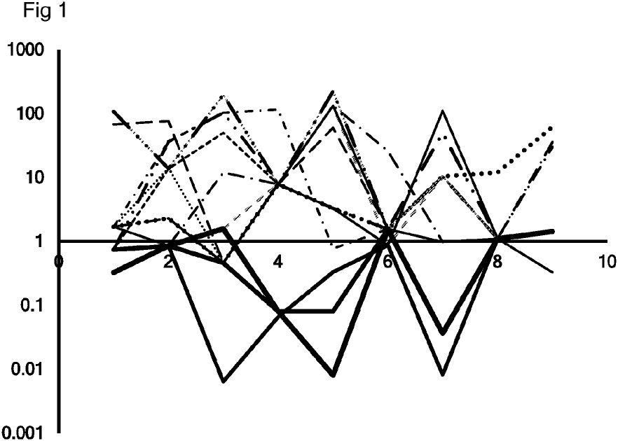

FIG. 1 shows tiling of a sub-centiMorgan area into short local haplotypes. Haplotype frequencies were used from a public dataset to accurately separate local African vs. European ancestry. FIG. 1 shows 9 windows of 12 markers were used. The likelihood of African local ancestry in AfrAm.

### Example 2: Local Ancestry and Comparative Calculations

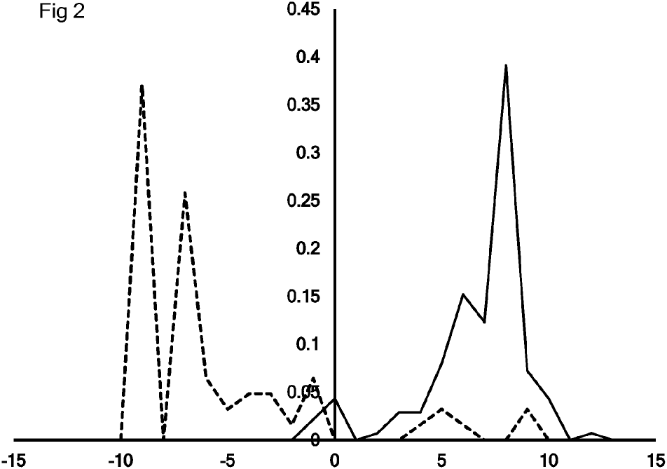

FIG. 2 shows that the IBS (Iberian / Spain) dataset, part of the 1000 Genomes (G1K) Europeans, exhibited up to 2-3 African haplotypes per locus in local ancestry mapping. Some European minor allele carriers in G1K had very African-like LD patterns. Eur (dotted line), Afr (solid line).

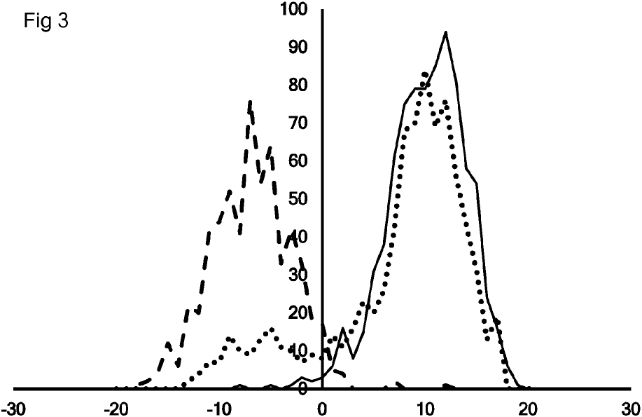

FIG. 3 shows a comparison of local/global ancestry distributions and allele frequencies (AF) were as expected. Distribution of predicted ethnic origin for rs 132390. Empirical AfrAm (dotted line), G1K Afr (solid line), G1K Eur (dashed line).

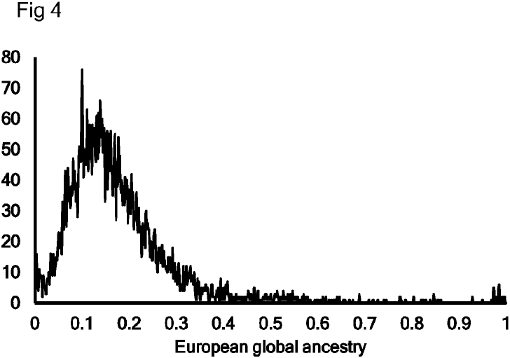

FIG. 4 shows European global ancestry averages were 17% with a wide distribution, as expected.

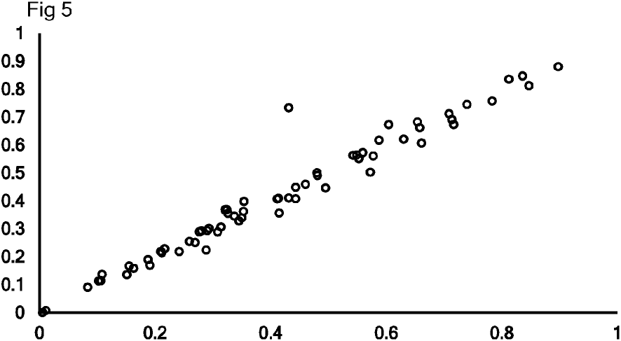

FIG. 5 shows empirical MAFs from European local ancestry segments (y axis) vs. G1K MAF values (x axis). FIG. 5 shows that local ancestry-derived empirical ancestral allele frequency (AF) estimates for the risk markers closely matched publicly available estimates of allele frequency in Europeans and Africans. Thus, the local ancestry calculations estimated with surprising accuracy the ancestral composition of the segments of the chromosome, and also assigned local ancestries to each risk allele.

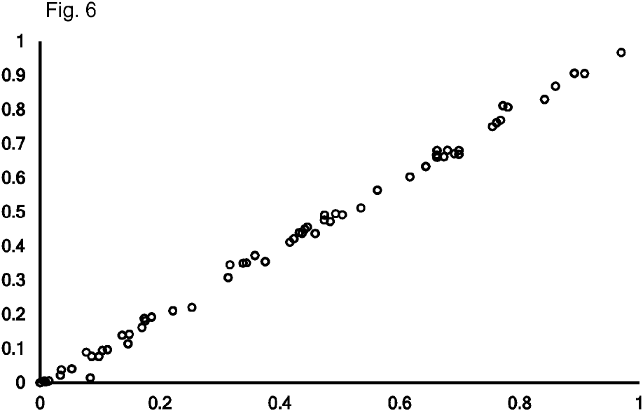

FIG. 6 shows empirical MAFs from African local ancestry segments (y axis) vs. G1K MAF values (x axis).

### Example 3: Calculating Odds of Local Ancestry

The odds of African vs. European ancestral origin of each of the haplotypes in each tile were calculated according to Equation I using empirical frequencies of haplotypes in the reference sets. Haplotypes that were absent in one of the reference sets were assigned to a frequency to 0.5 observations per set. Haplotypes absent in all of the reference sets were considered to be equally likely to be African vs. European.

The overall odds of African vs. European ancestral origin of the entire segment containing each of the alleles of a risk SNPs were calculated as the product of odds of all tile haplotypes contained within the segment. Fractional probabilities of each allele being “ancestrally European” vs. “ancestrally African,” or the fractional local ancestry LA, was found according to Equation II.

The overall fraction of European ancestry of each study subject, or the global ancestry GA, was calculated as the average of fractional European local ancestry for all loci, according to Equation III.

Incremental contributions of each allele to the polygenic risk score were calculated as local ancestry-specific risk effect (beta) multiplied by number of risk alleles genotyped less population-specific risk allele frequency, where GT_{SNP} = zero or 1.

The local ancestry ancestry-specific betas for the European local ancestry were obtained from Mavaddat (J Nat1 Cancer Inst., 2015, April 8, Vol. 107(5)). The local ancestry ancestry-specific betas for the African local ancestry were either taken directly from Feng (Cancer Epidemiol Biomarkers Prev., 2017, July, Vol. 26(7), pp. 1016-1026), or estimated from Feng and Wang (Sensors (Basel), 2017, July, Vol. 17(7), p. 1572). Estimating from Feng and Wang, for the nominally significant breast cancer risk associations, whenever consistent with the risk estimates of Mavaddat, the latter were assumed to be appropriate for African local ancestry as well, pan-ancestry risk markers. For the risk markers in Mavaddat that failed to show significant and consistent effects in the African studies, the African ancestry-specific betas were assumed to be equal to zero. Optionally, for the risk markers with marginally significant effects in African population study, the African ancestry-specific betas were assumed to be scaled down betas from the European study.

### Example 4: Polygenic Risk Scores

A comparative conventional polygenic risk score (cPRS) was calculated on the assumption that the respective betas of the ancestral groups β_{A,SNP} and β_{E,SNP} are interpolated using weights equal to respective global ancestry averages, GAA for the European ancestry and (1-GAA) for the African ancestry. The conventional score was centered on the admixed population-specific minor allele frequency MAF_{AA,SNP}, according to Equations IV and V.

A comparative, conventional method for global ancestry adjustment, which may interpolate the ancestral betas using the actual global ancestry of each subject, was calculated according to Equation VI.

Local ancestry adjustment was calculated, which may interpolate the ancestral betas using the fractional likelihood LA of each specific allele being European. Both ancestral components of the score were centered on respective ancestral minor allele frequencies MAF_{A,SNP} and MAF_{E,SNP}, according to Equation VII.

$$\begin{matrix}l o c a l P R S = \\ {\sum }_{S N P a l l e l e s} \left(\left(1 - L A\right) ∗ {β}_{A , S N P} ∗ \left(G {T}_{S N P} - M A {F}_{A , S N P}\right) +\right) \\ \left(L A ∗ {β}_{E , S N P} ∗ \left(G {T}_{S N P} - M A {F}_{E , S N P}\right)\right)\end{matrix} \qquad \text{Equation VII.}$$

The average African American population-specific beta was calculated by interpolation between European and African local ancestry-specific betas using average percentages of European vs. African ancestries. The global ancestry-corrected betas were calculated by interpolation between European and African local ancestry-specific betas using the actual percentage of European vs. African ancestry in each specific subject.

Methods of this invention can use any number of trait risk markers. Methods for determining a local ancestry-corrected polygenic risk score of this invention may use any 3 or more markers, or any 5 or more markers, or any 10 or more markers of a set of markers shown in Table 2.

### Example 5: Accuracy of Local Ancestry-Corrected Polygenic Risk Score for Breast Cancer Status

The local ancestry-corrected polygenic risk score (PRS) calculated by this invention was surprisingly more accurate that the conventional “global” ancestry-corrected PRS and the conventional average-ancestry-corrected PRS.

The association between polygenic risk score and the trait breast cancer status was evaluated by logistic regression adjusted for age and family history in a set of 4615 anonymized African American patients.

The association between polygenic risk score and the trait breast cancer status as calculated by the local ancestry-corrected method of this invention was surprisingly more accurate than for the conventional “global” ancestry-corrected method and the conventional “average” ancestry-corrected method.

Table 1 shows results that were obtained with a set of 64 risk markers, 19 of which were assumed to influence breast cancer risk in both European and African ancestral settings. As shown in Table 1, the methods for determining local ancestry-corrected polygenic risk score of this invention were surprisingly superior and more accurate than conventional “global ancestry-corrected” or “average ancestry-corrected” methods. The p-values in Table 1 show a greater than 3-fold enhancement by the methods of this invention for determining polygenic risk score with adjustment or correction by using local ancestry.

TABLE 1

Association between polygenic risk score and the trait breast cancer status as determined by this invention and comparative methods

| Method | Beta (PRS) | Std error (PRS) | p-value | N | Odds Ratio | Lower CI | Upper CI |
|---|---|---|---|---|---|---|---|
| cPRS⁽¹⁾ | 0.181 | 0.0423 | 1.71E-05 | 4615 | 1.20 | 1.10 | 1.30 |
| Global⁽²⁾ | 0.186 | 0.0429 | 1.22E-05 | 4615 | 1.20 | 1.11 | ---- |
| Local⁽³⁾ | 0.192 | 0.0424 | 5.54E-06 | 4615 | 1.21 | 1.11 | 1.32 |

(1) cPRS: conventional average-ancestry-corrected polygenic risk score
(2) Global: “global” ancestry-corrected polygenic risk score
(3) Local: local ancestry-corrected polygenic risk score of this invention

Table 2 shows the set of 64 risk markers.

TABLE 2

SNP VARIANT
rs7726159
rs10069690
rs2736108
rs2588809
rs999737
rs10759243
rs865686
rs2981579
rs11199914
rs7072776
rs13387042
rs11552449
rs11249433
rs4973768
rs889312
rs2046210
rs13281615
rs1011970
rs2380205
rs10995190
rs704010
rs4849887
rs1550623
rs6762644
rs12493607
rs9790517
rs6828523
rs1353747
rs1432679
rs11242675
rs204247
rs720475
rs9693444
rs6472903
rs11780156
rs7904519
rs3903072
rs11820646
rs12422552
rs17356907
rs11571833
rs2236007
rs941764
rs17817449
rs13329835
rs527616
rs3817198
rs10771399
rs1292011
rs3803662
rs6504950
rs8170
rs2363956
rs2823093
rs17879961
rs616488
rs1436904
rs4808801
rs3760982
rs6001930
rs4245739
rs6678914
rs12710696
rs11075995

### Example 6: Markers for Local Ancestry

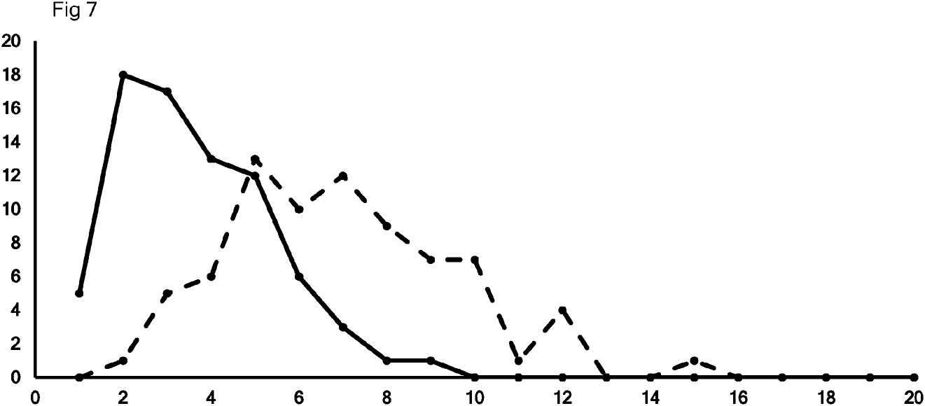

FIG. 7 shows a number of markers may be needed for Eur/Afr local ancestry assignment. A relatively small number of flanking markers (x axis) can be used to achieve surprisingly high 90% to 95% accuracy of local Afr/Eur ancestry identification. 95% accuracy (dashed line), 90% accuracy (solid line).

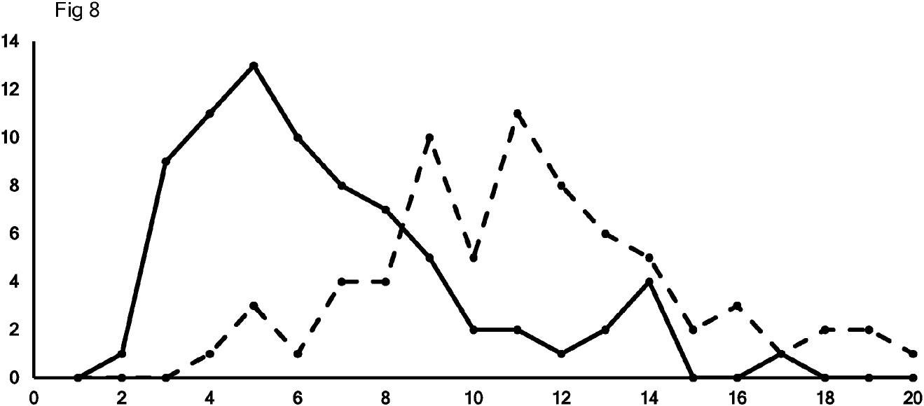

FIG. 8 shows a number of markers may be needed for Eur/EAS local ancestry assignment. A relatively small number of flanking markers (x axis) can be used to achieve surprisingly high 90% to 95% accuracy of separating European from East Asian ancestry identification. 95% accuracy (dashed line), 90% accuracy (solid line).

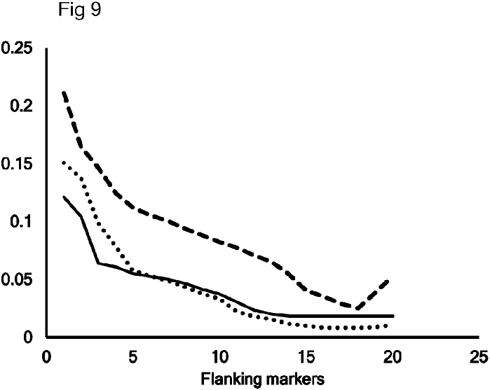

FIG. 9 shows an error rate of local ancestry assignment with unphased genotypes. FIG. 9 shows results with 3 SNP loci, rs258809, rs10759243, and rs1550623, and the y-axis displays the error rates of local ancestry deconvolution depending on the number of flanking loci.

What is claimed is:

1. A method for assessing a biological trait in a subject, the method comprising:
measuring a genotype in a sample from the subject, the genotype having windows centered on trait risk markers for the trait, wherein the windows comprise additional ancestry-informative markers flanking the risk markers;
phasing the genotype to determine haplotypes in each window using reference populations having admixed ancestries;
calculating the odds of a local ancestral origin for each window; and
calculating a polygenic risk score for the biological trait in the subject using the trait risk markers and the odds of a local ancestral origin for each window.

2. The method of claim 1, wherein the local ancestral origins of the windows are determined using the additional ancestry-informative markers.

3. The method of claim 1, wherein the number of trait risk markers is from 1-10,000.

4. The method of claim 1, wherein calculating the odds of a local ancestral origin comprises dividing the phased genotypes within each window into consecutive nonoverlapping tiles of up to about 300 additional ancestry-informative markers each, and calculating the odds of ancestral origin of each of the haplotypes in each tile using empirical frequencies of haplotypes in the reference population.

5. The method of claim 4, wherein each tile comprises 1-100 of the additional ancestry-informative markers.

6. The method of claim 4, wherein each tile comprises 5-20 of the additional ancestry-informative markers.

7. The method of claim 1, wherein the windows are about 1 MB in width.

8. The method of claim 1, wherein the genotype is determined by NGS.

9. The method of claim 1, wherein the genotype is determined with a sequencing chip.

10. The method of claim 1, wherein the biological trait is cancer likelihood.

11. The method of claim 1, wherein the genomic risk markers are cancer markers.

12. The method of claim 1, wherein the additional ancestry-informative markers are SNP markers or indel markers.

13. The method of claim 1, wherein the genomic risk markers are breast cancer SNP markers.

14. The method of claim 1, wherein the calculating a polygenic risk score comprises calculating incremental contributions of each allele to the polygenic risk score as a local ancestry-specific risk effect beta multiplied by a number of risk alleles genotyped, which is zero or 1, less a population-specific risk allele frequency.

15. A method for recommending therapy for a subject having a disease, the method comprising:
measuring a genotype in a sample from the subject, the genotype having windows centered on trait risk markers for the trait, wherein the windows comprise additional ancestry-informative markers flanking the risk markers;
phasing the genotype to determine haplotypes in each window using reference populations having admixed ancestries;
calculating the odds of a local ancestral origin for each window;
calculating a polygenic risk score for the biological trait in the subject using the trait risk markers and the odds of a local ancestral origin for each window; and
recommending a therapy for the disease based on the risk score indicating a need for the therapy.

16. The method of claim 15, wherein the disease is cancer.

17. The method of claim 15, wherein the disease is breast cancer.

18. The method of claim 15, wherein the therapy is one of:
a therapy for the disease;
a monitoring period followed by a therapy for the disease;
a tapering of a therapy for the disease.

19. The method of claim 15, wherein the therapy is one or more of surgery, cryoablation, radiation therapy, bone marrow transplant, chemotherapy, immunotherapy, hormone therapy, stem cell therapy, drug therapy, biological therapy, and administration of a pharmaceutical, prophylactic or therapeutic compound.

20. A method for identifying a subject having a disease who benefits from a treatment, the method comprising:
measuring a genotype in a sample from the subject, the genotype having windows centered on trait risk markers for the trait, wherein the windows comprise additional ancestry-informative markers flanking the risk markers;
phasing the genotype to determine haplotypes in each window using reference populations having admixed ancestries;
calculating the odds of a local ancestral origin for each window;
calculating a polygenic risk score for the biological trait in the subject using the trait risk markers and the odds of a local ancestral origin for each window; and
identifying the subject having the disease who benefits from the treatment for the disease based on the risk score indicating a need for the treatment.

21. The method of claim 20, wherein the disease is cancer.

22. The method of claim 20, wherein the disease is breast cancer.

23. The method of claim 20, wherein the treatment is one of:
a therapy for the disease;
a monitoring period followed by a therapy for the disease;
a tapering of a therapy for the disease.

24. The method of claim 20, wherein the treatment is one or more of surgery, cryoablation, radiation therapy, bone marrow transplant, chemotherapy, immunotherapy, hormone therapy, stem cell therapy, drug therapy, biological therapy, and administration of a pharmaceutical, prophylactic or therapeutic compound.

25. A method for treating a disease in a subject in need thereof, the method comprising:
measuring a genotype in a sample from the subject, the genotype having windows centered on trait risk markers for the trait, wherein the windows comprise additional ancestry-informative markers flanking the risk markers;
phasing the genotype to determine haplotypes in each window using reference populations having admixed ancestries;
calculating the odds of a local ancestral origin for each window; and
calculating a polygenic risk score for the biological trait in the subject using the trait risk markers and the odds of a local ancestral origin for each window, wherein the polygenic risk score indicates a need for treating the subject; and
administering to the subject one of:
a therapy for the disease;
a monitoring period followed by a therapy for the disease;
a tapering of a therapy for the disease.

26. The method of claim 25, wherein the therapy is a cancer therapy selected from one or more of surgery, cryoablation, radiation therapy, bone marrow transplant, chemotherapy, immunotherapy, hormone therapy, stem cell therapy, drug therapy, biological therapy, and administration of a pharmaceutical, prophylactic or therapeutic compound.

27. A method for monitoring a response of a subject having a disease, the method comprising:
measuring a genotype in a sample from the subject, the genotype having windows centered on trait risk markers for the trait, wherein the windows comprise additional ancestry-informative markers flanking the risk markers;
phasing the genotype to determine haplotypes in each window using reference populations having admixed ancestries;
calculating the odds of a local ancestral origin for each window; and
calculating a polygenic risk score for the biological trait in the subject using the trait risk markers and the odds of a local ancestral origin for each window.

28. A method for prognosing a subject having a disease, the method comprising:
measuring a genotype in a sample from the subject, the genotype having windows centered on trait risk markers for the trait, wherein the windows comprise additional ancestry-informative markers flanking the risk markers;
phasing the genotype to determine haplotypes in each window using reference populations having admixed ancestries;
calculating the odds of a local ancestral origin for each window;
calculating a polygenic risk score for the biological trait in the subject using the trait risk markers and the odds of a local ancestral origin for each window; and
prognosing the subject as having a poor prognosis for the disease based on the risk score.

29. A system for assessing risk of a disease in a subject, the system comprising:
a processor for receiving genomic data from a sample of the subject;
one or more processors for carrying out the steps:
measuring a genotype in a sample from the subject, the genotype having windows centered on trait risk markers for the trait, wherein the windows comprise additional ancestry-informative markers flanking the risk markers;
phasing the genotype to determine haplotypes in each window using reference populations having admixed ancestries;
calculating the odds of a local ancestral origin for each window; and
calculating a polygenic risk score for the biological trait in the subject using the trait risk markers and the odds of a local ancestral origin for each window; and
a display for displaying and/or reporting the risk score.

30. A non-transitory machine-readable storage medium having stored therein instructions for execution by a processor which cause the processor to perform the steps of a method for assessing risk of a disease in a subject, the method comprising:
receiving genomic data from a sample from the subject;
measuring a genotype in the sample, the genotype having windows centered on trait risk markers for the trait, wherein the windows comprise additional ancestry-informative markers flanking the risk markers;
phasing the genotype to determine haplotypes in each window using reference populations having admixed ancestries;
calculating the odds of a local ancestral origin for each window;
calculating a polygenic risk score for the biological trait in the subject using the trait risk markers and the odds of a local ancestral origin for each window; and
sending to a processor output for displaying and/or reporting the risk score.
